(19) United States
(12) Patent Application Publication
(10) Pub. No.: US 2024/0100357 A1
(43) Pub. Date: Mar. 28, 2024

(54) METHOD AND APPARATUS TO MODIFY DOSE VALUES DURING RADIATION TREATMENT PLANNING

(71) Applicant: Siemens Healthineers International AG, Steinhausen (CH)

(72) Inventors: Jani Kurki, Helsinki (FI); Matti Kemppainen, Jarvenpaa (FI); Laura Korhonen, Helsinki (FI); Veli-Pekka Kihniä, Espoo (FI); Lauri Halko, Helsinki (FI); Jarmo Makkonen, Helsinki (FI); Pekka Sorvari, Espoo (FI)

(21) Appl. No.: 17/953,584

(22) Filed: Sep. 27, 2022

(51) Int. Cl. A61N 5/10

(52) U.S. Cl. CPC A61N 5/103; A61N 5/1049

(57) ABSTRACT

A control circuit configured as a multi-criteria optimizer generates a radiation therapy treatment plan. The circuit then determines a resultant dose distribution as a function of the radiation therapy treatment plan and presents, on a display, at least a part of that resultant dose distribution. The latter may comprise, for example, presenting at least one isodose line on the display. The control circuit then detects user manipulation of a user interface. By one approach, this manipulation does not include corresponding immediate movement of any part of the presented resultant dose distribution itself. In particular, the detected user manipulation does not result in dragged movement of an isodose line on the display. Upon detecting that user manipulation, the control circuit modifies a dose value at a particular location on the presented resultant dose distribution as a function of that user manipulation of the user interface to provide a modified dose distribution and/or, by one approach, a new candidate radiation therapy treatment plan as an interpolation and/or mix of the treatment plans generated by a multi-criteria optimizer.

### TECHNICAL FIELD

These teachings relate generally to treating a patient's planning target volume with energy pursuant to an energy-based treatment plan and more particularly to optimizing an energy-based treatment plan.

### BACKGROUND

The use of energy to treat medical conditions comprises a known area of prior art endeavor. For example, radiation therapy comprises an important component of many treatment plans for reducing or eliminating unwanted tumors. Unfortunately, applied energy does not inherently discriminate between unwanted material and adjacent tissues, organs, or the like that are desired or even critical to continued survival of the patient. As a result, energy such as radiation is ordinarily applied in a carefully administered manner to at least attempt to restrict the energy to a given target volume. A so-called radiation treatment plan often serves in the foregoing regards.

A radiation treatment plan typically comprises specified values for each of a variety of treatment-platform parameters during each of a plurality of sequential fields. Treatment plans for radiation treatment sessions are often automatically generated through a so-called optimization process. As used herein, “optimization” will be understood to refer to improving a candidate treatment plan without necessarily ensuring that the optimized result is, in fact, the singular best solution. Such optimization often includes automatically adjusting one or more physical treatment parameters (often while observing one or more corresponding limits in these regards) and mathematically calculating a likely corresponding treatment result (such as a level of dosing) to identify a given set of treatment parameters that represent a good compromise between the desired therapeutic result and avoidance of undesired collateral effects.

Multicriteria optimization comprises one approach to such optimization. Generally speaking, multicriteria optimization employs more than one objective function to be optimized simultaneously with one another. In a typical application setting, at least two of those objective functions conflict with one another. As a result, optimizing a radiation treatment plan in this way typically involves making trade-offs between two or more conflicting objectives. The applicant has determined that it can be difficult for a user to properly envision and/or to explore the corresponding solution space.

### BRIEF DESCRIPTION OF THE DRAWINGS

The above needs are at least partially met through provision of the method and apparatus to modify dose values during radiation treatment planning described in the following detailed description, particularly when studied in conjunction with the drawings, wherein:

FIG. 1 comprises a block diagram as configured in accordance with various embodiments of these teachings;

FIG. 2 comprises a flow diagram as configured in accordance with various embodiments of these teachings;

FIG. 3 comprises a screen shot as configured in accordance with various embodiments of these teachings;

FIG. 4 comprises a screen shot as configured in accordance with various embodiments of these teachings;

FIG. 5 comprises a screen shot as configured in accordance with various embodiments of these teachings;

FIG. 6 comprises a screen shot as configured in accordance with various embodiments of these teachings;

FIG. 7 comprises a screen shot as configured in accordance with various embodiments of the invention;

FIG. 8 comprises a screen shot as configured in accordance with various embodiments of these teachings; and

FIG. 9 comprises a screen shot as configured in accordance with various embodiments of the invention.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present teachings. Also, common but well-understood elements that are useful or necessary in a commercially feasible embodiment are often not depicted in order to facilitate a less obstructed view of these various embodiments of the present teachings. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein. The word “or” when used herein shall be interpreted as having a disjunctive construction rather than a conjunctive construction unless otherwise specifically indicated.

### DETAILED DESCRIPTION

Generally speaking, pursuant to these various embodiments, a control circuit configured as a multi-criteria optimizer generates a radiation therapy treatment plan. The control circuit then determines a resultant dose distribution as a function of the radiation therapy treatment plan and presents, on a display, at least a part of that resultant dose distribution. The latter may comprise, for example, presenting at least one isodose line on the display. The control circuit then detects user manipulation of a user interface. By one approach, this manipulation does not include corresponding immediate movement of any part of the presented resultant dose distribution itself. In particular, the detected user manipulation does not result in dragged movement of an isodose line on the display. Upon detecting that user manipulation, the control circuit modifies a dose value at a particular location on the presented resultant dose distribution as a function of that user manipulation of the user interface to provide a modified dose value.

By one approach, the aforementioned user interface comprises a mouse interface. By one approach the mouse interface includes a scroll wheel. In the latter case, detecting the user manipulation of the user interface can comprise, at least in part, detecting user manipulation of the scroll wheel. In such a case, and by one approach, manipulating the scroll wheel in a first direction of rotation leads to increasing the dose value at the aforementioned particular location on the presented resultant dose distribution while manipulating the scroll wheel in a second direction of rotation that is opposite to the first direction of rotation leads to decreasing the dose value at the aforementioned particular location on the presented resultant dose distribution.

These teachings are flexible in practice and will accommodate, for example, presenting on the display at least one of: the aforementioned modified dose value, a corresponding modified dose distribution, and a dose dependent statistic. As another example, these teachings will accommodate automatically determining when the user's manipulation of the user interface results in an unpermitted dose value and responsively, when such is true, not modifying the dose value at the particular location on the presented resultant dose distribution (or, in the alternative, only modifying the dose value to the extent permitted and no further). These teachings will also accommodate providing an alert regarding the unpermitted dose value situation as well.

By one approach, these teachings will also accommodate reconsidering the aforementioned radiation therapy treatment plan as a function of the modified dose value. This reconsideration can comprise any of a variety of predetermined responses.

So configured, these teachings help facilitate a user being able to explore trade-offs and/or otherwise adjust a dose in a multi-criteria optimization setting in a manner that is highly intuitive and that requires little training. The foregoing functionality can be achieved with very little overhead system requirements, time requirements, or delays.

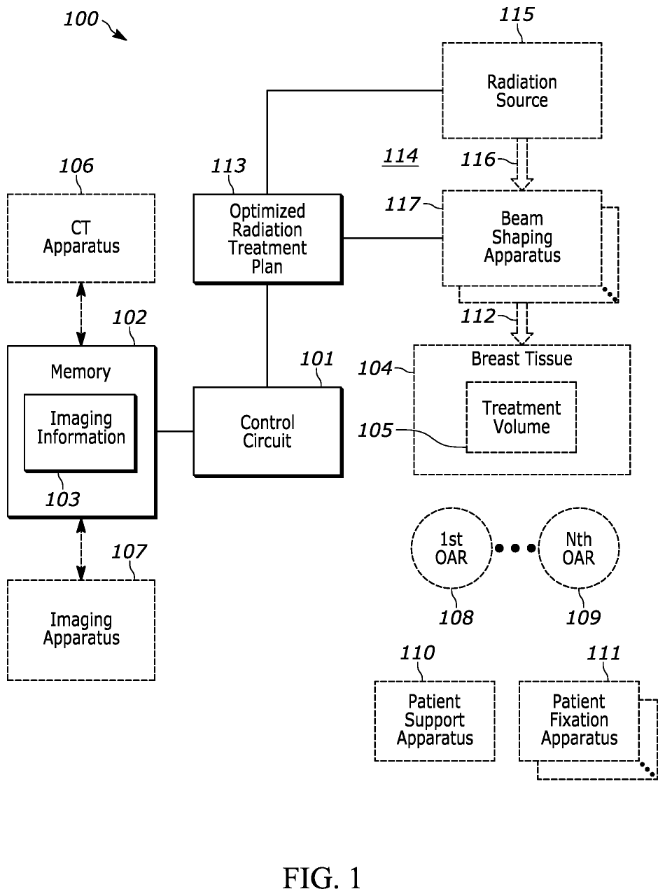

These and other benefits may become clearer upon making a thorough review and study of the following detailed description. Referring now to the drawings, and in particular to FIG. 1, an illustrative apparatus 100 that is compatible with many of these teachings will first be presented.

In this particular example, the enabling apparatus 100 includes a control circuit 101. Being a “circuit,” the control circuit 101 therefore comprises structure that includes at least one (and typically many) electrically-conductive paths (such as paths comprised of a conductive metal such as copper or silver) that convey electricity in an ordered manner, which path(s) will also typically include corresponding electrical components (both passive (such as resistors and capacitors) and active (such as any of a variety of semiconductor-based devices) as appropriate) to permit the circuit to effect the control aspect of these teachings.

Such a control circuit 101 can comprise a fixed-purpose hard-wired hardware platform (including but not limited to an application-specific integrated circuit (ASIC) (which is an integrated circuit that is customized by design for a particular use, rather than intended for general-purpose use), a field-programmable gate array (FPGA), and the like) or can comprise a partially or wholly-programmable hardware platform (including but not limited to microcontrollers, microprocessors, and the like). These architectural options for such structures are well known and understood in the art and require no further description here. This control circuit 101 is configured (for example, by using corresponding programming as will be well understood by those skilled in the art) to carry out one or more of the steps, actions, and/or functions described herein.

The control circuit 101 operably couples to a memory 102. This memory 102 may be integral to the control circuit 101 or can be physically discrete (in whole or in part) from the control circuit 101 as desired. This memory 102 can also be local with respect to the control circuit 101 (where, for example, both share a common circuit board, chassis, power supply, and/or housing) or can be partially or wholly remote with respect to the control circuit 101 (where, for example, the memory 102 is physically located in another facility, metropolitan area, or even country as compared to the control circuit 101).

In addition to information such as optimization information for a particular patient and information regarding a particular radiation treatment platform as described herein, this memory 102 can serve, for example, to non-transitorily store the computer instructions that, when executed by the control circuit 101, cause the control circuit 101 to behave as described herein. (As used herein, this reference to “non-transitorily” will be understood to refer to a non-ephemeral state for the stored contents (and hence excludes when the stored contents merely constitute signals or waves) rather than volatility of the storage media itself and hence includes both non-volatile memory (such as read-only memory (ROM) as well as volatile memory (such as a dynamic random access memory (DRAM).)

By one optional approach the control circuit 101 also operably couples to a user interface 103. This user interface 103 can comprise any of a variety of user-input mechanisms (such as, but not limited to, keyboards and keypads, cursor-control devices (including but not limited to a mouse having, for example, one or more selection buttons and a scroll wheel), touch-sensitive displays, speech-recognition interfaces, gesture-recognition interfaces, and so forth) and/or user-output mechanisms (such as, but not limited to, visual displays, audio transducers, printers, and so forth) to facilitate receiving information and/or instructions from a user and/or providing information to a user.

If desired the control circuit 101 can also operably couple to a network interface (not shown). So configured the control circuit 101 can communicate with other elements (both within the apparatus 100 and external thereto) via the network interface. Network interfaces, including both wireless and non-wireless platforms, are well understood in the art and require no particular elaboration here.

By one approach, a computed tomography apparatus 106 and/or other imaging apparatus 107 as are known in the art can source some or all of any desired patient-related imaging information.

In this illustrative example the control circuit 101 is configured to ultimately output an optimized energy-based treatment plan (such as, for example, an optimized radiation treatment plan 113). This energy-based treatment plan typically comprises specified values for each of a variety of treatment-platform parameters during each of a plurality of sequential exposure fields. In this case the energy-based treatment plan is generated through an optimization process, examples of which are provided further herein.

By one approach the control circuit 101 can operably couple to an energy-based treatment platform 114 that is configured to deliver therapeutic energy 112 to a corresponding patient 104 in accordance with the optimized energy-based treatment plan 113. These teachings are generally applicable for use with any of a wide variety of energy-based treatment platforms/apparatuses. In a typical application setting the energy-based treatment platform 114 will include an energy source such as a radiation source 115 of ionizing radiation 116.

By one approach this radiation source 115 can be selectively moved via a gantry along an arcuate pathway (where the pathway encompasses, at least to some extent, the patient themselves during administration of the treatment). The arcuate pathway may comprise a complete or nearly complete circle as desired. By one approach the control circuit 101 controls the movement of the radiation source 115 along that arcuate pathway, and may accordingly control when the radiation source 115 starts moving, stops moving, accelerates, de-accelerates, and/or a velocity at which the radiation source 115 travels along the arcuate pathway.

As one illustrative example, the radiation source 115 can comprise, for example, a radio-frequency (RF) linear particle accelerator-based (linac-based) x-ray source. A linac is a type of particle accelerator that greatly increases the kinetic energy of charged subatomic particles or ions by subjecting the charged particles to a series of oscillating electric potentials along a linear beamline, which can be used to generate ionizing radiation (e.g., X-rays) 116 and high energy electrons.

A typical energy-based treatment platform 114 may also include one or more support apparatuses 110 (such as a couch) to support the patient 104 during the treatment session, one or more patient fixation apparatuses 111, a gantry or other movable mechanism to permit selective movement of the radiation source 115, and one or more energy-shaping apparatuses (for example, beam-shaping apparatuses 117 such as jaws, multi-leaf collimators, and so forth) to provide selective energy shaping and/or energy modulation as desired.

In a typical application setting, it is presumed herein that the patient support apparatus 110 is selectively controllable to move in any direction (i.e., any X, Y, or Z direction) during an energy-based treatment session by the control circuit 101. As the foregoing elements and systems are well understood in the art, further elaboration in these regards is not provided here except where otherwise relevant to the description.

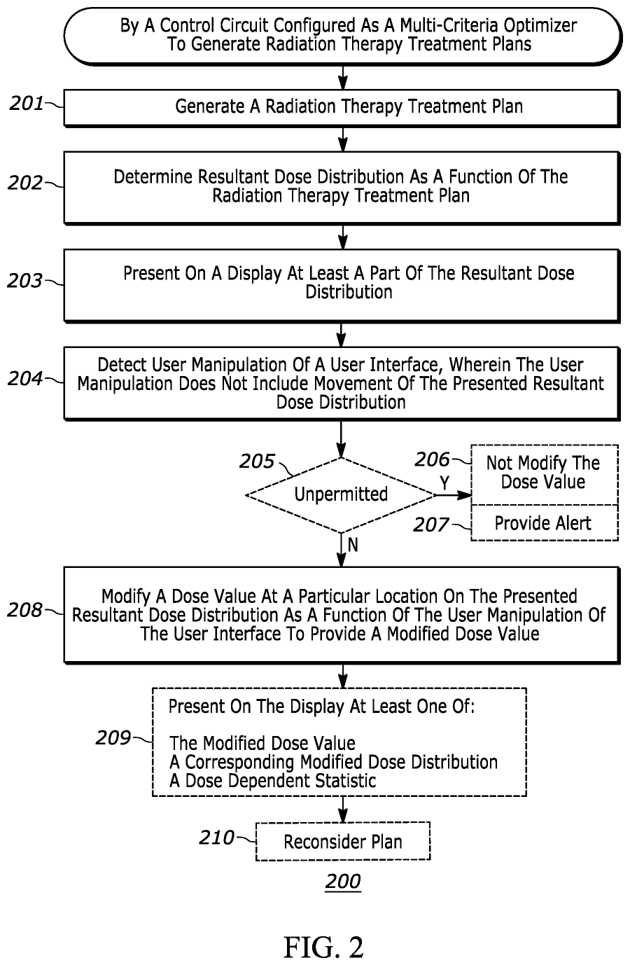

Referring now to FIG. 2, a process 200 that can be carried out, for example, in conjunction with the above-described application setting (and more particularly via the aforementioned control circuit 101 in conjunction with the aforementioned user interface 103) will be described. Generally speaking, this process 200 serves to facilitate generating an optimized radiation treatment plan 113 to thereby facilitate treating a particular patient with therapeutic radiation using a particular radiation treatment platform per that optimized radiation treatment plan.

For the sake of an illustrative example, it is presumed here that the control circuit 101 is configured as a multi-criteria optimizer to generate radiation therapy treatment plans. (Multi-criteria optimization is known in the art. See, for example, U.S. patent application publication number 2017/0072221 (entitled KNOWLEDGE BASED MULTI-CRITERIA OPTIMIZATION FOR RADIOTHERAPY TREATMENT PLANNING), the contents of which are fully incorporated herein by this reference. Accordingly, further details in these regards are generally not provided herein for the sake of brevity.)

At block 201, the control circuit 101 generates a radiation therapy treatment plan. These teachings are not overly sensitive to any particular approaches in these regards and will accommodate any of a variety of approaches to generating such a plan.

At block 202, the control circuit 101 determines a resultant dose distribution as a function of the radiation therapy treatment plan. Again, these teachings are not overly sensitive to any particular approaches in these regards and will accommodate any of a variety of approaches to determining such distribution. In a typical application setting, this resultant dose distribution will include the radiation dose as distributed both through the target volume as well as one or more organs-at-risk and/or surrounding tissue.

At block 203, the control circuit 101 presents on a display (such as a display that comprises a part of the aforementioned user interface 103) at least a part of the aforementioned resultant dose distribution. These teachings will again accommodate any of a variety of approaches in these regards. By one such approach, and as an illustrative example, the dose distribution can be presented, at least in part, as a plurality of isodose lines. Such lines may or may not be differentiated by color and may or may not be presented in conjunction with a text field presenting the corresponding dosage.

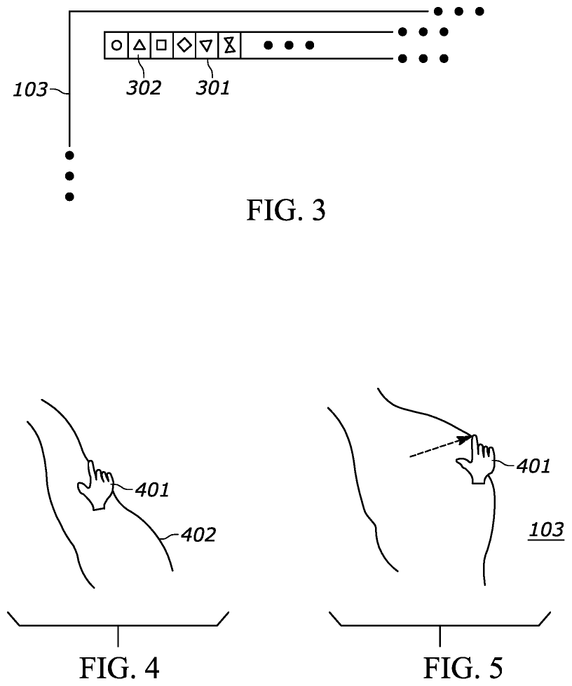

By one approach, and referring momentarily to FIG. 3, the display of the user interface 103 can present a toolbar 301. That toolbar 301 can provide a variety of user-selectable tools that can be used, for example, during the planning/optimization activities referred to herein. Each such tool may be represented in the toolbar 301 by a corresponding icon. One such icon 302 can serve, for example, to bring up a user-interface tool that facilitates modifying the aforementioned resultant dose distribution as described herein.

By one approach, and as illustrated in FIG. 4, this dose distribution modification tool can provide, in one optional mode of operation, a corresponding cursor 401 (such as the illustrated pointing finger cursor) on the user interface 103 display. Using this cursor 401, the user can employ a mouse to left click on a given isodose line 402 and then, as shown in FIG. 5, drag that selected portion of that isodose line 402 to a new location to thereby modify the resultant dose distribution. (Further details regarding such an approach can be found in U.S. Pat. No. 11,235,172 entitled Radiation Treatment Planning Real-Time User Interface, the contents of which are fully incorporated herein by this reference.) This dose distribution modification tool may also, however, provide an alternative approach in these regards.

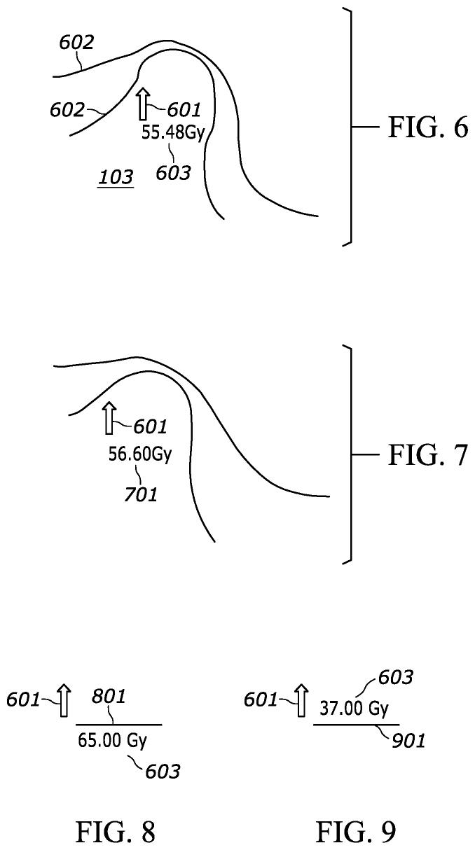

In particular, and referring now to block 204 of FIG. 2, these teachings provide for a different approach to modifying the aforementioned resultant dose distribution. At block 204, the control circuit detects a different user manipulation of the user interface 103 (in particular and in this example, the user-interface tool that is activated by the aforementioned icon 302 in the aforementioned toolbar 301). As an illustrative example, and without intending to suggest any limitations in these regards, this particular user-interface tool may be characterized by a uniquely corresponding cursor such as the upwardly-pointing long-body arrow denoted by reference numeral 601 in FIG. 6. In such a case, the detected user manipulation can be conducted in concert with this uniquely-corresponding arrow cursor.

Pursuant to these teachings, although this activity can result in modification of the aforementioned resultant dose distribution, this user manipulation does not include current movement of any part of the presented resultant dose distribution. For example, the user manipulation of the user interface 103 does not result in dragged movement of the aforementioned at least one isodose line on the display. (To be clear, one or more displayed isodose lines may eventually be re-presented in a new position, but such repositioning is not a concurrent result of dragging the line via the user interface.)

By one approach, the aforementioned user interface 103 comprises a mouse interface that interacts with the aforementioned uniquely-corresponding cursor. More particularly, and by one approach, that mouse interface includes a scroll wheel as described above. So configured, the aforementioned detection of the user manipulation of the user interface 103 comprises, at least in part, user manipulation of that scroll wheel.

Pursuant to these teachings, such manipulation of the scroll wheel serves to modify a dose value at a particular location on the presented resultant dose distribution. That particular location can correspond, for example, to the tip of the arrow-shaped cursor 601 in FIG. 6. It should be noted that, by one approach, the particular location may or may not coincide with a presented isodose line 602 at the whim of the user. By another approach, if desired, the particular location may be automatically selected as a closest point on a closest isodose line to the tip of the cursor arrow 601.

By one approach, upon activating this tool, a text field 603 accompanies the cursor that provides the user with information regarding the current dose at the location that corresponds to the tip of the cursor arrow. As the user manipulates the mouse to move the location of the cursor about the displayed dose distribution, that current dose information will change accordingly. It may be noted that the dose information displayed in this manner can be different from any dose information that might also be displayed with any of the presented isodose lines.

For the sake of an illustrative example, it will be assumed here that such manipulation also increases that local dose value when the user manipulates the scroll wheel in a first direction of rotation (such as clockwise) and decreases that dose value when the user manipulates the scroll wheel in a second direction of rotation (such as counterclockwise) that is opposite to the first direction of rotation. FIG. 7 provides an illustrative example where the user has manipulated the mouse's scroll wheel to increase the local dose, and the corresponding presented dose amount has been increased to 56.60 Gy as denoted by reference numeral 701.

By one approach, an adjusted dose value remains at the last attained value unless the user again manipulates the scroll wheel while selecting that particular location. By another approach, the user may be required to click with the mouse to accept a given attained value (otherwise, the modified dose value will revert to its original value once the user moves the cursor to a different location). These teachings will also accommodate, for example, undoing a previous dose modification using the scroll wheel in combination with a different command.

Additional details regarding these teachings will now be provided by way of some examples. It will be understood that the specific details of these examples are intended to serve an illustrative purpose and are not to be understood as suggesting any particular limitations with respect to these teachings.

At optional block 205, the control circuit 101 can be configured to automatically determine when the aforementioned user's manipulation of the user interface 103 results in an unpermitted dose value, such as a dose value that exceeds some threshold in either a negative or positive direction. That threshold may represent an absolute value or it may be a relative value, such as a certain percentage of the starting point dosage. Also, if desired, that threshold may vary depending, for example, upon whether the particular location corresponds to a treatment volume or to an organ-at-risk. In some cases, these teachings will provide for detecting a situation when no plan or plan mix is found in an accessed plan collection where the user-proposed dose value at the selected location can be met. Using this approach, the lower/upper limits are the smallest/largest doses that can be found at this location amongst the plans in the accessed collection of radiation treatment plans.

Upon detecting such a condition, at block 206 the control circuit 101 can optionally not modify the dose value at the particular location on the presented resultant dose distribution notwithstanding the user's attempt to change the dose value. In the alternative, upon detecting such a condition, the control circuit 101 can optionally modify the dose value, but not beyond, for example, the corresponding threshold value or some other previously-established limit.

In lieu of the foregoing or in combination therewith, and as illustrated at optional block 207, upon detecting such a condition, the control circuit 101 can provide an alert regarding the unpermitted dose value. That alert may be a visually-perceivable alert and/or an audible alert as desired. As one illustrative approach in these regards, and referring momentarily to FIGS. 8 and 9, an upper bar 801 can be displayed over the dose text field 603 to alert the user that the local dose has reaching a maximum permitted value, while a lower bar 901 can be displayed under the dose text field 603 to alert the user that the local dose has reached a minimum permitted value. These teachings will of course accommodate a variety of other approaches, including the use of differing colors, flashing pixels, highlighting, alternative fonts, and so forth to provide alert-based indicia.

When such is not the case, or when the optional activity just described is not utilized, at block 208 the control circuit 101 modifies the dose value at the particular location on the presented resultant dose distribution as a function of the user manipulation of the user interface 101 to provide a modified dose value. That modification may be achieved via, for example, further calculations and/or accessing other information such as other previously optimized radiation treatment plans. For example, this step can comprise selecting one treatment plan or interpolated/mixed plan from amongst the radiation therapy treatment plans that are generated by the multi-criteria optimizer.

At optional block 209, these teachings will accommodate optionally presenting resultant modified information on the aforementioned display. In addition to presenting the modified dose value itself as described above, the control circuit 101 may present a corresponding modified dose distribution. In particular, accommodating the modified dose value will often result in modifying other dose values and those collateral modifications may be presented via, for example, the presentation of modified isodose lines. And again in lieu of the foregoing or in combination therewith, these teachings will accommodate having the control circuit 101 present on the display one or more dose dependent statistics.

Should the user again manipulate, in this example, the aforementioned scroll wheel while this particular tool is engaged, the activities described above in blocks 204 through 209 can be repeated to reflect further adjustments to the corresponding dose value.

At optional block 210, these teachings will accommodate reconsidering the radiation treatment plan as a function, at least in part, of the aforementioned modified dose value. These teachings will accommodate a variety of approaches in such regards. By one approach, the modified dose value(s) selected by the user might serve to automatically adjust corresponding optimization objectives, following which the control circuit 101 may completely reoptimize the radiation treatment plan.

In the case where the user makes the above-description modification(s) as a real-time modification in a multi-criteria optimization workspace, the control circuit 101 can reconsider the radiation treatment plan by, at least in part, selecting a new, possibly interpolated plan from a plan collection where the modified constraint is met. Selecting that new plan will typically result in changing the corresponding dose distribution (and dose volume histograms as well). In many application settings, no further optimization per se is required in that workspace. (Upon completing plan exploration in the multi-criteria optimization workspace, however, in some application settings, such as volumetric modulated arc therapy, the control circuit 101 may then perform a more time-consuming re-optimization to yield a machine deliverable radiation treatment plan, where the guiding dose volume histograms and three-dimensional dosing requirements are met at least closely. In other application settings, such as intensity-modulated radiation therapy, the plan may not be re-optimized, but optimal fluence may be computed as a linear combination of the optimal fluences for at least some of the plans in the plan collection, following which a final multi-leaf collimator leaf sequence can be generated.)

Once the radiation treatment plan is finalized, these teachings will accommodate then using that plan 113 to treat the patient with radiation using, for example, the aforementioned radiation treatment platform 114.

Those skilled in the art will recognize that a wide variety of modifications, alterations, and combinations can be made with respect to the above-described embodiments without departing from the scope of the invention, and that such modifications, alterations, and combinations are to be viewed as being within the ambit of the inventive concept.

What is claimed is:

1. A method comprising:
by a control circuit configured as a multi-criteria optimizer to generate radiation therapy treatment plans:
generating a radiation therapy treatment plan;
determining resultant dose distribution as a function of the radiation therapy treatment plan;
presenting on a display at least a part of the resultant dose distribution;
detecting user manipulation of a user interface, wherein the user manipulation does not include movement of the presented resultant dose distribution;
modifying a dose value at a particular location on the presented resultant dose distribution as a function of the user manipulation of the user interface to provide a modified dose value.

2. The method of claim 1 wherein the radiation therapy treatment plan comprises a photon therapy treatment plan.

3. The method of claim 1 wherein presenting on the display at least a part of the resultant dose distribution comprises, at least in part, presenting at least one isodose line on the display.

4. The method of claim 3 wherein the user manipulation of the user interface does not result in dragged movement of the at least one isodose line on the display.

5. The method of claim 1 wherein the user interface comprises a mouse interface.

6. The method of claim 5 wherein the mouse interface includes a scroll wheel and wherein detecting user manipulation of the user interface comprises, at least in part, user manipulation of the scroll wheel.

7. The method of claim 6 wherein modifying the dose value at a particular location on the presented resultant dose distribution as a function of the user manipulation of the user interface comprises increasing the dose value when the user manipulates the scroll wheel in a first direction of rotation and decreasing the dose value when the user manipulates the scroll wheel in a second direction of rotation that is opposite to the first direction of rotation.

8. The method of claim 1 further comprising:
presenting on the display at least one of:
the modified dose value;
a corresponding modified dose distribution; and
a dose dependent statistic.

9. The method of claim 1 further comprising:
automatically determining when the user's manipulation of the user interface results in an unpermitted dose value and responsively, when such is true, not modifying the dose value at the particular location on the presented resultant dose distribution and providing an alert regarding the unpermitted dose value.

10. The method of claim 1 further comprising:
reconsidering the radiation therapy treatment plan as a function of the modified dose value.

11. An apparatus comprising:
a display;
a user interface;
a control circuit operably coupled to the display and to the user interface and configured as a multi-criteria optimizer to generate radiation therapy treatment plans, wherein the control circuit is further configured to:
generate a radiation therapy treatment plan;
determine resultant dose distribution as a function of the radiation therapy treatment plan;
present on the display at least a part of the resultant dose distribution;
detect user manipulation of the user interface, wherein the user manipulation does not include movement of the presented resultant dose distribution;
modify a dose value at a particular location on the presented resultant dose distribution as a function of the user manipulation of the user interface to provide a modified dose value.

12. The apparatus of claim 11 wherein the radiation therapy treatment plan comprises a photon therapy treatment plan.

13. The apparatus of claim 11 wherein the control circuit is configured to present on the display at least a part of the resultant dose distribution by, at least in part, presenting at least one isodose line on the display.

14. The apparatus of claim 13 wherein the control circuit is further configured such that the user manipulation of the user interface does not result in dragged movement of the at least one isodose line on the display.

15. The apparatus of claim 11 wherein the user interface comprises a mouse interface.

16. The apparatus of claim 15 wherein the mouse interface includes a scroll wheel and wherein the control circuit is configured to detect the user manipulation of the user interface by detecting, at least in part, user manipulation of the scroll wheel.

17. The apparatus of claim 16 wherein the control circuit is configured to modify the dose value at a particular location on the presented resultant dose distribution as a function of the user manipulation of the user interface by increasing the dose value when the user manipulates the scroll wheel in a first direction of rotation and decreasing the dose value when the user manipulates the scroll wheel in a second direction of rotation that is opposite to the first direction of rotation.

18. The apparatus of claim 11 wherein the control circuit is further configured to:
present on the display at least one of:
the modified dose value;
a corresponding modified dose distribution; and
a dose dependent statistic.

19. The apparatus of claim 11 wherein the control circuit is further configured to:
automatically determine when the user's manipulation of the user interface results in an unpermitted dose value and responsively, when such is true, not modify the dose value at the particular location on the presented resultant dose distribution and provide an alert regarding the unpermitted dose value.

20. The apparatus of claim 11 wherein the control circuit is configured to:
reconsider the radiation therapy treatment plan as a function of the modified dose value.
